(19) United States
(12) Plant Patent Application Publication
(10) Pub. No.: US 2014/0189912 P1
(43) Pub. Date: Jul. 3, 2014

(54) Corylus plant named 'DORRIS'

(50) ;

(71) Applicant: State of Oregon acting by and through the State Board of Higher Education on behalf of Oregon State University, (US)

(72) Inventors: Shawn A. Mehlenbacher, Corvallis, OR (US); David C. Smith, Corvallis, OR (US); Rebecca L. McCluskey, Corvallis, OR (US)

(73) Assignees: State University, Corvallis, OR (US); State of Oregon acting by and through the State Board of Higher Education of behalf of Oregon

(21) Appl. No.: 13/694,675

(22) Filed: Dec. 24, 2012

(51) Int. Cl. A01H 5/00

(52) U.S. Cl. PLT/152

(57) ABSTRACT

A new and distinct cultivar of Corylus plant named ‘Dorris’ characterized by a spreading plant habit and low vigor, yellowish-green developing and fully expanded leaves during the spring and summer, resistance to eastern filbert blight caused by the fungus Anisogramma anomala (Peck) E. Müller, presence of random amplified polymorphic DNA markers 152-800 and 268-580, expression of incompatibility alleles S₁ and S₁₂ in the styles, and DNA fingerprints at 14 of 24 microsatellite marker loci differ from both parents OSU 309.074 and ‘Delta’, and from one parent at an additional 9 marker loci.

### ACKNOWLEDGMENT OF GOVERNMENT SUPPORT

This invention was made with government support under Specific Cooperative Agreement No. 58-5358-4542 awarded by the United States Department of Agriculture. The government has certain rights in the invention.

### BOTANICAL DENOMINATION

Corylus avellana

### VARIETY DESIGNATION

‘Dorris’

### BACKGROUND

The present invention relates to a new and distinct cultivar of Corylus plant, (hazelnut, filbert) botanically known as Corylus avellana, and hereinafter referred to by the name ‘Dorris’. Corylus avellana is in the family Betulaceae.

The new Corylus resulted from a controlled cross of female parent OSU 309.074 (unpatented) and male parent ‘Delta’ (unpatented) made in 1997 by Shawn A. Mehlenbacher and David C. Smith. Hybrid seeds from the cross were harvested in August 1997, stratified, and seedlings grown in the greenhouse during the summer of 1998. From this cross, a total of 307 seedling trees were planted in the field in Corvallis, Oreg., USA in October, 1998. ‘Dorris’ was discovered and selected by the Inventors as a single plant within the progeny of the stated cross-pollination in a controlled environment in Corvallis, Oreg.

‘Dorris’ was originally assigned the designation OSU 876.041, which indicates the row and tree location of the original seedling. OSU 309.074 is from a cross of ‘Tonda Gentile delle Langhe’ (unpatented)×OSU 23.017 (unpatented). ‘Tonda Gentile delle Langhe’ is an important cultivar in Piemonte, northern Italy. OSU 23.017 is from a cross of ‘Barcelona’ (unpatented)×‘Extra Ghiaghli’ (unpatented). ‘Extra Ghiaghli’, obtained from Greece, is a clone of the important Turkish cultivar ‘Tombul’ (unpatented). ‘Delta’ was released by the Oregon Agricultural Experiment Station in 2002.

The new cultivar was asexually reproduced by rooted suckers annually for eight years (2003-2010) in Corvallis, Oreg. The new cultivar was also asexually propagated by whip grafting in 2004 in Corvallis, Oreg. The unique features of this new Corylus are stable and reproduced true-to-type in successive generations of asexual reproduction.

### SUMMARY OF THE INVENTION

The following traits have been repeatedly observed and are determined to be the unique characteristics of ‘Dorris’. These characteristics in combination distinguish ‘Dorris’ as a new and distinct cultivar:

1. Spreading plant habit and low vigor.

2. Yellowish-green developing and fully expanded leaves during the spring and summer.

3. Resistance to eastern filbert blight caused by the fungus Anisogramma anomala (Peck) E. Muller.

4. Presence of random amplified polymorphic DNA markers 152-800 and 268-580 in DNA of ‘Dorris’ amplified by the polymerase chain reaction. These two markers are linked to a dominant allele for resistance to eastern filbert blight from the cultivar Gasaway (unpatented).

5. Expression of incompatibility alleles S₁ and S₁₂ in the styles,

6. DNA fingerprints at 14 of 24 microsatellite marker loci differ from both parents OSU 309.074 and ‘Delta’, and from one parent at an additional 9 marker loci. The microsatellite primers are shown in Table 1, and allele sizes are shown in Table 2. DNA fingerprints of grandparent ‘Tonda Gentile delle Langhe’ and great-grandparents ‘Barcelona’ and ‘Extra Ghiaghli’ are also shown in attached Table 2.

In comparisons in two replicated trials conducted in Corvallis, Oreg., plants of the new Corylus differed from plants of the Corylus avellana cultivar Barcelona (unpatented), and other cultivars and selections of Corylus avellana known to the Inventors primarily in nut size, nut shape, kernel percentage (ratio of kernel weight to nut weight), frequency of blank nuts (nuts lacking kernels), time of pollen shed, time of nut maturity, length of the husk or involucre, and plant size.

### BRIEF DESCRIPTION OF THE DRAWINGS

The accompanying colored photographs illustrate the overall appearance of the new cultivar, showing the colors as true as it is reasonably possible to obtain in colored reproductions of this type. Foliage colors in the photographs may differ slightly from the color values cited in the detailed botanical description which accurately describe the colors of the new Corylus.

FIG. 1 shows a tree of the new cultivar ‘Dorris’ growing in a field in the summer, in Corvallis, Oreg.

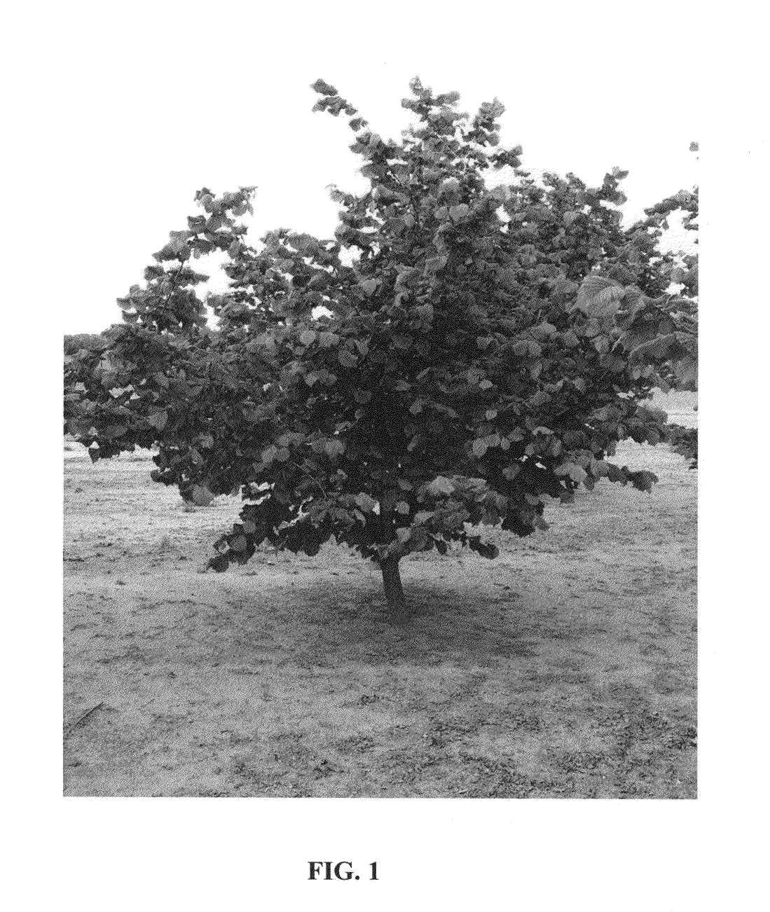

FIG. 2 shows the tree of the new cultivar ‘Dorris’ growing in a field in January, in Corvallis, Oreg.

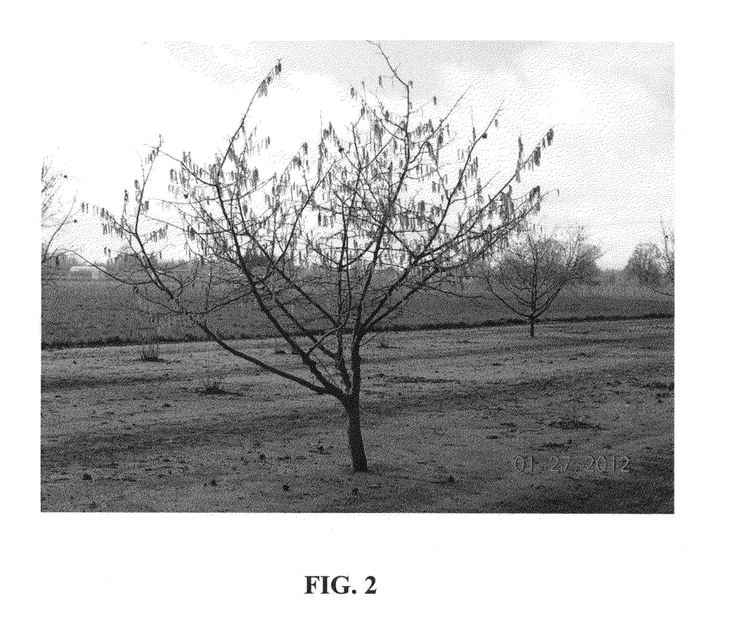

FIG. 3 shows typical nuts, raw kernels, and blanched kernels of ‘Dorris’ hazelnut compared to those of ‘Jefferson’ hazelnut.

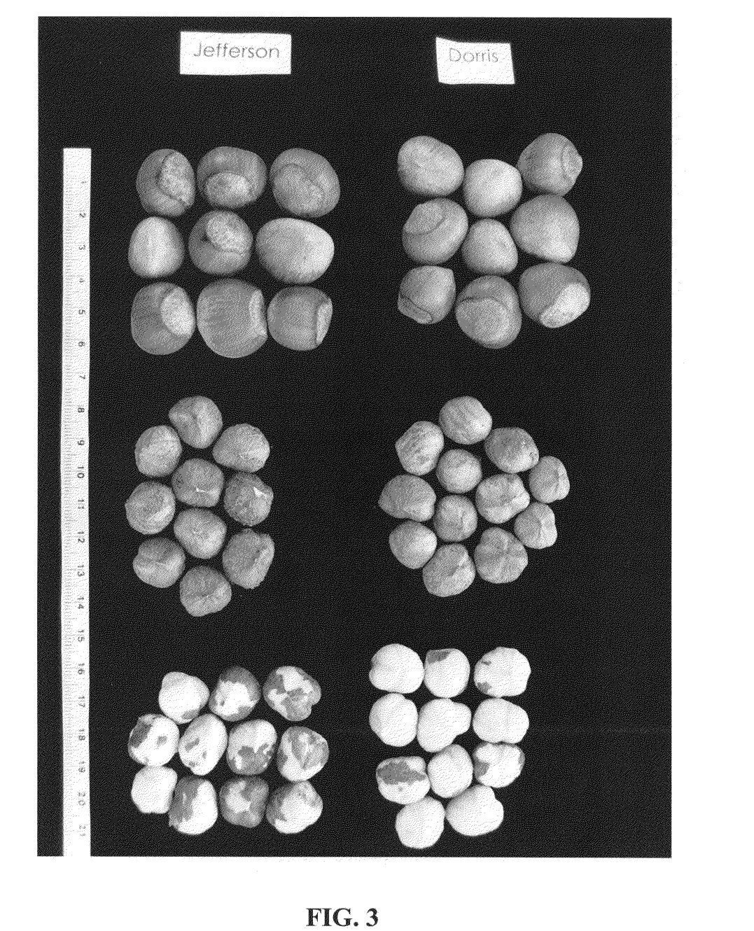

FIG. 4 shows husks of ‘Dorris’ hazelnut tree.

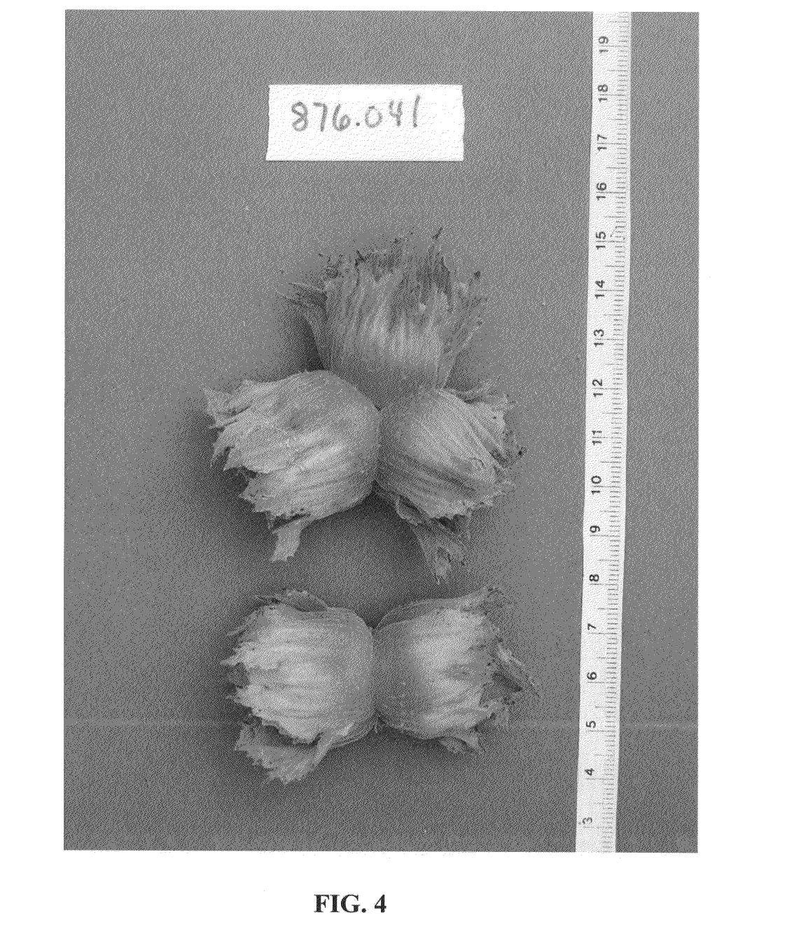

FIG. 5 shows the typical nuts, raw kernels, and blanched kernels of ‘Dorris’ hazelnut compared to those of ‘Barcelona’ hazelnut and other hazelnut cultivars.

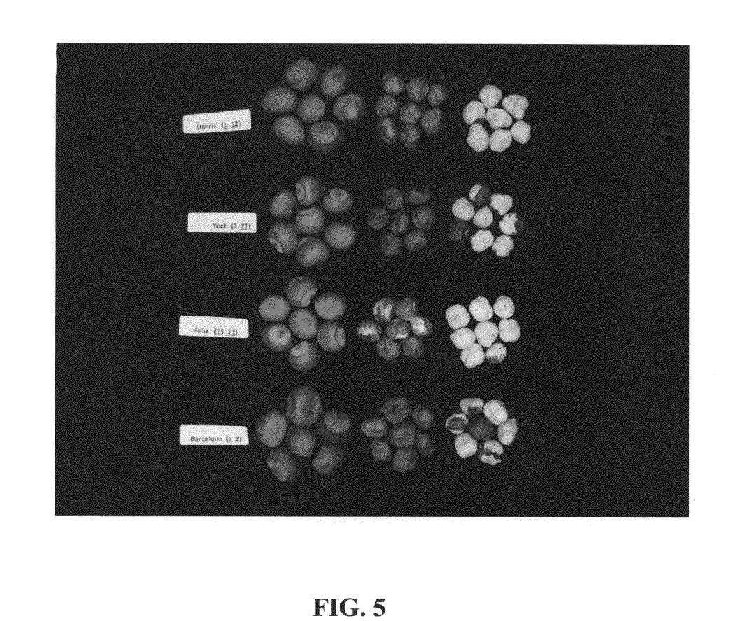

### DETAILED PLANT DESCRIPTION

The cultivar Dorris has not been observed under all possible environmental conditions. The phenotype may vary somewhat with variations in environment such as temperature and light intensity, without, however, any variance in genotype. The aforementioned photographs and following observations and measurements describe plants grown in Corvallis, Oreg. under commercial practice outdoors in the field during the fall, winter and spring. Plants used for the photographs and description were propagated by tie-off layerage and growing on their own roots, and about seven years old. In the following description, color references are made to The Royal Horticultural Society Colour Chart, 1966 Edition, except where general terms of ordinary dictionary significance are used.
Botanical classification: Corylus avellana cultivar Dorris.
Parentage:
   Female, or seed, parent.—Corylus avellana selection 309.074 (unpatented).
   Male, or pollen, parent.—Corylus avellana cultivar Delta (unpatented).
Propagation: (type rooted suckers):
   Time to initiate roots.—about 30 days at 20° C.
   Time to produce a rooted young plant.—about six months at 22° C.
   Root description.—fine to thick; freely branching; creamy white in color.
Propagation (type whip grafting):
   Time to budbreak on the scions.—bout 14 days at 25° C.
   Time to produce a grafted plant.—about six months at 25° C.
Plant description:
   General appearance.—perennial shrub. Spreading plant habit.
   Growth and branching habit.—freely branching; about 15 lateral branches develop per plant. Pinching, i.e., removal of the terminal apices, enhances branching with lateral branches potentially forming at every node.
   Size.—Plant height is about 4 meters; plant diameter or spread is about 5 meters.
   Vigor.—low vigor growth habit.
Lateral branch description:
   Length.—about 32 cm.
   Diameter.—about 6 mm.
   Internode length.—about 3.0 cm.
   Texture.—smooth, glabrous.
   Strength.—strong.
   Color.—immature — 152B; mature — 152B.
Foliage description:
   Arrangement.—alternate, simple.
   Length.—about 10.2 cm.
   Width.—about 9.1 cm.
   Shape.—oblong to ovate.
   Apex.—obtuse to acute.
   Base.—cordate.
   Margin.—serrate.
   Texture.—upper and lower surfaces — slightly pubescent.
   Venation pattern.—pinnate.
   Color.—Developing foliage, upper surface 144A, lower surfaces: 187A. Fully expanded foliage, upper surface: Spring and summer, 143A; late summer and fall, 143A. Fully expanded foliage, lower surface: Spring and summer, 139C; late summer and fall, 139C. Venation, upper surface: Spring and summer, 139C; late summer and fall, 139C. Venation, lower surface: Spring and summer, 139D; late summer and fall, 139D.
Petiole description:
   Length.—about 2.7 cm.
   Diameter.—about 1.8 mm.
   Texture.—upper and lower surfaces — pubescent.
   Color.—upper surface: Spring and summer, 139D; late summer and fall, 139D. lower surface: Spring and summer, 139D; late summer and fall, 139D.
Flower description:
   Male inflorescences.—catkins, color prior to elongation 194C. Female inflorescence — style color 048B.
Nut description:
   Length.—about 19.1 mm.
   Width.—about 20.7 mm.
   Depth.—about 18.2 mm.
   Nut shape.—round.
   Nut shape index [(width+depth)/2*Length].—1.02.
   Nut compression index (width/depth).—1.14.
   Nut shell color.—164B.
   Nut weight.—about 3.35 grams.
   Kernel weight.—about 1.40 grams.
   Kernel percentage (kernel weight/nut weight).—about 43%.
Disease/pest resistance: Plants of the new Corylus are highly resistant to eastern filbert blight caused by the fungus Anisogramma anomala (Peck) E. Müller. Plants of the new Corylus are highly resistant to bud mites (Phytoptus avellanae Nal.), while plants of ‘Tonda Gentile delle Langhe’ are highly susceptible, and plants of ‘Barcelona’ are highly resistant.
Temperature tolerance: tolerates temperatures from −10 to 38° C. in the field in Corvallis, Oreg.

TABLE 1
Primers and annealing temperatures for the 24 microsatellite marker loci
used to fingerprint ‘Dorris’ and other hazelnut cultivars

| | Repeat | | | | | | | | | Primers | Primers | |
|---|---|---|---|---|---|---|---|---|---|---|---|---|
| Locus | Motif | Size | Tₐ | n | He | Ho | PIC | r | LG | 5′-3′ | 5′-3′ | Reference |
| A613 | (TC)₁₃ | 149- | 60 | 14 | 0.85 | 0.85 | 0.85 | 0.00 | 11 | Ned- | R- | Gurcan et al. |
| | (CA)₁₂ | 177 | | | | | | | | CACACGCCTT | CCCCTTTCACA | 2010 |
| | | | | | | | | | | GTCACTCTTT | TGTTTGCTT | |
| | | | | | | | | | | (SEQ ID NO: | (SEQ ID NO: | |
| | | | | | | | | | | 1) | 2) | |
| A614 | (TC)₁₇ | 125- | 60 | 14 | 0.85 | 0.85 | 0.84 | 0.00 | 6 | Hex- | R- | Gurcan et al. |
| | (CA)₁₀ | 156 | | | | | | | | TGGCAGAGCT | GCAGTGGAGGA | 2010 |
| | NNN(CA)₆ | | | | | | | | | TTGTCAGCTT | TTGCTGACT | |
| | | | | | | | | | | (SEQ ID NO: | (SEQ ID NO: | |
| | | | | | | | | | | 3) | 4) | |
| A616 | (AC)₁₁ | 136- | 60 | 13 | 0.85 | 0.85 | 0.83 | 0.00 | 8 | Fam- | R- | Gurcan et al. |
| | | 162 | | | | | | | | CACTCATACC | ATGGCTTTTGC | 2010 |
| | | | | | | | | | | GCAAACTCCA | TTCGTTTTG | |
| | | | | | | | | | | (SEQ ID NO: | (SEQ ID NO: | |
| | | | | | | | | | | 5) | 6) | |
| A640 | (CT)₁₅ | 354- | 67 | 11 | 0.80 | 0.73 | 0.7 | 0.04 | 10 | F- | Fam- | Gurcan et al. |
| | (CA)₁₃ | 378 | | | | | | | | TGCCTCTGCA | CGCCATATAAT | 2010 |
| | | | | | | | | | | GTTAGTCATC | TGGGATGCTTG | |
| | | | | | | | | | | AAATGTAGG | TTG | |
| | | | | | | | | | | (SEQ ID NO: | (SEQ ID NO: | |
| | | | | | | | | | | 7) | 8) | |
| B107 | (CT)₁₄ | 112- | 55 | 14 | 0.85 | 0.80 | 0.83 | 0.02 | 10 | Ned- | R- | Boccacci et |
| | | 151 | | | | | | | | GTAGGTGCAC | AACACCATATT | al. 2005; |
| | | | | | | | | | | TTGATGTGCT | GAGTCTTTCAA | Gokirmak et |
| | | | | | | | | | | TTAC | AGC | al. 2009 |
| | | | | | | | | | | (SEQ ID NO: | (SEQ ID NO: | |
| | | | | | | | | | | 9) | 10) | |
| B617 | (GA)₁₅ | 280- | 60 | 9 | 0.80 | 0.78 | 0.78 | 0.01 | 8 | Fam- | R- | Gurcan et al. |
| | | 298 | | | | | | | | TCCGTGTTGA | TGTTTTTGGTG | 2010 |
| | | | | | | | | | | GTATGGACGA | GAGCGATG | |
| | | | | | | | | | | (SEQ ID NO: | (SEQ ID NO: | |
| | | | | | | | | | | 11) | 12) | |
| B619 | (TC)₂₁ | 146- | 60 | 14 | 0.88 | 0.88 | 0.7 | 0.00 | 3 | Fam- | R- | Gurcan et al. |
| | | 180 | | | | | | | | AGTCGGCTCC | GCGATCTGACC | 2010 |
| | | | | | | | | | | CCTTTTCTC | TCATTTTTG | |
| | | | | | | | | | | (SEQ ID NO: | (SEQ ID NO: | |
| | | | | | | | | | | 13) | 14) | |
| B634 | (AG)₁₅ | 218- | 60 | 9 | 0.76 | 0.76 | 0.73 | 0.00 | 4 | Hex- | R- | Gurcan et al. |
| | | 238 | | | | | | | | CCTGCATCCA | GTGCAGAGGTT | 2010 |
| | | | | | | | | | | GGACTCATTA | GCACTCAAA | |
| | | | | | | | | | | (SEQ ID NO: | (SEQ ID NO: | |
| | | | | | | | | | | 15) | 16) | |
| B657 | (AG)₁₅ | 210- | 60 | 8 | 0.84 | 0.98 | 0.82 | −0.08 | 11 | Ned- | R- | Gurcan et al. |
| | | 228 | | | | | | | | GAGAGTGCGT | AGCCTCACCTC | 2010 |
| | | | | | | | | | | CTTCCTCTGG | CAACGAAC | |
| | | | | | | | | | | (SEQ ID NO: | (SEQ ID NO: | |
| | | | | | | | | | | 17) | 18) | |
| B671 | (AG)6NN | 221- | 60 | 13 | 0.86 | 0.88 | 0.84 | −0.01 | 9 | Hex- | R- | Gurcan et al. |
| | (GA)₁₇ | 249 | | | | | | | | TTGCCAGTGC | ACCAGCTCTGG | 2010 |
| | | | | | | | | | | ATACTCTGATG | GCTTAACAC | |
| | | | | | | | | | | (SEQ ID NO: | (SEQ ID NO: | |
| | | | | | | | | | | 19) | 20) | |
| B709 | (GA)₂₁ | 219- | 60 | 8 | 0.74 | 0.76 | 0.70 | −0.01 | 5 | Ned- | R- | Gurcan et al. |
| | | 233 | | | | | | | | CCAAGCACGA | GCGGGTTCTCG | 2010 |
| | | | | | | | | | | ATGAACTCAA | TTGTACACT | |
| | | | | | | | | | | (SEQ ID NO: | (SEQ ID NO: | |
| | | | | | | | | | | 21) | 22) | |
| B733 | (TC)₁₅ | 161- | 60 | 8 | 0.68 | 0.68 | 0.63 | 0.00 | 7.2 | Ned- | R- | Gurcan et al. |
| | | 183 | | | | | | | | CACCCTCTTC | CATCCCCTGTT | 2010 |
| | | | | | | | | | | ACCACCTCAT | GGAGTTTTC | |
| | | | | | | | | | | (SEQ ID NO: | (SEQ ID NO: | |
| | | | | | | | | | | 23) | 24) | |
| B741 | (GT)₅ | 176- | 60 | 10 | 0.77 | 0.78 | 0.74 | 0.00 | 5 | Fam- | R- | Gurcan et al. |
| | (GA)₁₂ | 194 | | | | | | | | GTTCACAGGC | CGTGTTGCTCA | 2010 |
| | | | | | | | | | | TGTTGGGTTT | TGTGTTGTG | |
| | | | | | | | | | | (SEQ ID NO: | (SEQ ID NO: | |
| | | | | | | | | | | 25) | 26) | |
| B749 | (TC)₁₂ | 200- | 60 | 6 | 0.60 | 0.64 | 0.51 | −0.03 | 1 | Hex- | R- | Gurcan et al. |
| | | 210 | | | | | | | | GGCTGACAAC | TCGGCTAGGGT | 2010 |
| | | | | | | | | | | ACAGCAGAAA | TAGGGTTTT | |
| | | | | | | | | | | (SEQ ID NO: | (SEQ ID NO: | |
| | | | | | | | | | | 27) | 28 | |
| B751 | (GA)₁₅ | 141- | 60 | 7 | 0.80 | 0.80 | 0.77 | 0.01 | 7.2 | Fam- | R- | Gurcan et al. |
| | | 153 | | | | | | | | AGCTGGTTCT | AAACTCAAATA | 2010 |
| | | | | | | | | | | TCGACATTCC | AAACCCCTGCTC | |
| | | | | | | | | | | (SEQ ID NO: | (SEQ ID NO: | |
| | | | | | | | | | | 29) | 30) | |
| B774 | (AG)₁₅ | 195- | 60 | 8 | 0.80 | 0.80 | 0.77 | 0.01 | 5 | Ned- | R- | Gurcan et al. |
| | | 213 | | | | | | | | GTTTTGCGAG | TGTGTGTGGTC | 2010 |
| | | | | | | | | | | CTCATTGTCA | TGTAGGCACT | |
| | | | | | | | | | | (SEQ ID NO: | (SEQ ID NO: | |
| | | | | | | | | | | 31) | 32) | |
| B776 | (GA)₁₇ | 134- | 60 | 7 | 0.71 | 0.60 | 0.67 | 0.07 | 6 | Fam- | R- | Gurcan et al. |
| | | 148 | | | | | | | | TGTATGTACA | TGAGGGGAAG | 2010 |
| | | | | | | | | | | CACGGAGAGA | AGGTTTGATG | |
| | | | | | | | | | | GAGA | (SEQ ID NO: | |
| | | | | | | | | | | (SEQ ID NO: | 34) | |
| | | | | | | | | | | 33) | | |
| B795 | (TC)₈ | 296- | 60 | 12 | 0.76 | 0.74 | 0.74 | 0.01 | NA | Fam- | R- | Gurcan et al. |
| | Ns(CT)₇ | 332 | | | | | | | | GACCCACAA | TGGGCATCATC | 2010 |
| | Ns(CT)₁₀ | | | | | | | | | ACAATAACCT | CAGGTCTA | |
| | Ns(TC)₅ | | | | | | | | | ATCTC | (SEQ ID NO: | |
| | | | | | | | | | | (SEQ ID NO: | 36) | |
| | | | | | | | | | | 35) | | |
| C115 | (TAA)₅ | 167- | 60 | 14 | 0.80 | 0.80 | 0.77 | 0.00 | 4 | Fam- | GTTTCCAGATC | Bassil et al. |
| | (GAA)₁₂ | 226 | | | | | | | | ATTTTCCGCA | TGCCTCCATAT | 2005b, |
| | | | | | | | | | | GATAATACAGG | AAT | Gokirmak et |
| | | | | | | | | | | (SEQ ID NO: | (SEQ ID NO: | al. 2009 |
| | | | | | | | | | | 37) | 38) | |
| KG809 | (AGG)₆ | 333- | 55 | 5 | 0.66 | 0.64 | 0.60 | 0.01 | 4 | Hex- | F- | Gurcan and |
| | | 345 | | | | | | | | AGGCATCAG | GGAAGGTGAG | Mehlenbacher |
| | | | | | | | | | | TTCATCCAA | AGAAATCAAGT | 2010 |
| | | | | | | | | | | (SEQ ID NO: | (SEQ ID NO: | |
| | | | | | | | | | | 39) | 40) | |
| KG811 | (GA)₁₇ | 240- | 58 | 12 | 0.83 | 0.82 | 0.81 | 0.01 | 2 | Ned- | F- | Gurcan and |
| | | 278 | | | | | | | | AAGGCGGCA | GAACAACTGAA | Mehlenbacher |
| | | | | | | | | | | CTCGCTCAC | GACAGCAAAG | 2010 |
| | | | | | | | | | | (SEQ ID NO: | (SEQ ID NO: | |
| | | | | | | | | | | 41) | 42) | |
| KG827 | (CT)₁₃AA | 264- | 67 | 9 | 0.78 | 0.84 | 0.75 | −0.04 | 9 | Fam- | GAGGGAGCAA | Gurcan and |
| | (CA)₇ | 282 | | | | | | | | AGAACTCCGA | GTCAAAGTTGA | Mehlenbacher |
| | | | | | | | | | | CTAATAATCC | GAAGAAA | 2010 |
| | | | | | | | | | | TAACCCTTGC | (SEQ ID NO: | |
| | | | | | | | | | | (SEQ ID NO: | 44) | |
| | | | | | | | | | | 43) | | |
| KG830 | (CT)₁₄ | 279- | 67 | 9 | 0.79 | 0.78 | 0.76 | 0.00 | 9 | Ned- | AAAGCAACTCA | Gurcan and |
| | GTATT | 311 | | | | | | | | TGGAGGAAGT | TAGCTGAAGTC | Mehlenbacher |
| | (CA)₈ | | | | | | | | | TTTGAATGGT | CAATCA | 2010 |
| | | | | | | | | | | AGTAGAGGA | (SEQ ID NO: | |
| | | | | | | | | | | (SEQ ID NO: | 46) | |
| | | | | | | | | | | 45) | | |
| Soman- | (AAT)₅ | | 54 | 3 | 0.60 | 0.98 | 0.51 | −0.27 | NA | Hex- | R- | unpublished |
| G | | | | | | | | | | TGGCGTTGCA | GCCATCTTTAG | |
| | | | | | | | | | | ACATATTCTC | AAAGTTCGATA | |
| | | | | | | | | | | (SEQ ID NO: | CAG | |
| | | | | | | | | | | 47) | (SEQ ID NO: | |
| | | | | | | | | | | | 48) | |

Primer fluorescent tags are FAM, HEX, and NED.
Ta: annealing temperature (° C.)
N: number of alleles
He: expected heterozygosity
Ho: observed heterozygosity
PIC: polymorphism information content
r: estimated null allele frequency
LG: linkage group

TABLE 2
Allele sizes in ‘Dorris’ and other hazelnut cultivars at 24 microsatellite loci.

| | | | | | ‘Tonda Gentile |
|---|---|---|---|---|---|
| Tag | Locus | ‘Dorris’ | ‘309.074’ | ‘Delta’ | delle Langhe’ |
| NED | A613 | 149/167 | 157/167 | 149/177 | 151/157 |
| HEX | A614 | 132/158 | 125/132 | 143/158 | 125/135 |
| FAM | A616 | 148/150 | 148/158 | 150/150 | 148/150 |
| FAM | A640 | 372/374 | 368/374 | 362/372 | 354/368 |
| NED | B107 | 112/122 | 112/152 | 122/130 | 134/152 |
| FAM | B617 | 286/296 | 294/296 | 286/286 | 286/296 |
| FAM | B619 | 156/164 | 164/174 | 156/164 | 148/164 |
| HEX | B634 | 226/226 | 226/226 | 226/234 | 226/226 |
| NED | B657 | 210/226 | 210/226 | 222/226 | 218/226 |
| HEX | B671 | 227/247 | 227/237 | 235/247 | 237/241 |
| NED | B709 | 227/227 | 227/227 | 227/227 | 227/227 |
| NED | B733 | 171/179 | 171/173 | 173/179 | 171/173 |
| FAM | B741 | 177/186 | 177/177 | 177/186 | 177/184 |
| HEX | B749 | 206/206 | 206/208 | 206/208 | 206/208 |
| FAM | B751 | 143/151 | 143/153 | 143/151 | 149/153 |
| NED | B774 | 203/207 | 203/203 | 207/213 | 203/211 |
| FAM | B776 | 137/137 | 137/137 | 137/150 | 137/137 |
| FAM | B795 | 330/330 | 330/330 | 314/330 | 312/330 |
| FAM | C115 | 194/215 | 173/194 | 197/215 | 173/173 |
| HEX | KG809 | 336/345 | 336/339 | 345/345 | 336/339 |
| NED | KG811 | 254/264 | 242/254 | 254/264 | 254/264 |
| FAM | KG827 | 270/282 | 268/282 | 270/270 | 266/268 |
| NED | KG830 | 295/297 | 291/295 | 291/297 | 291/295 |
| HEX | SMNG | 196/200 | 196/200 | 196/196 | 196/200 |
| | | | | ‘Extra | |
| | | Tag | ‘Barcelona’ | Ghiaghli’ | ‘Gasaway’ |
| | | NED | 151/159 | 167/169 | 159/161 |
| | | HEX | 125/131 | 125/150 | 143/158 |
| | | FAM | 142/150 | 150/158 | 148/148 |
| | | FAM | 354/374 | 374/374 | 362/368 |
| | | NED | 112/134 | 116/116 | 122/128 |
| | | FAM | 286/290 | 294/296 | 292/296 |
| | | FAM | 156/170 | 164/174 | 170/174 |
| | | HEX | 226/226 | 226/226 | 220/232 |
| | | NED | 218/222 | 210/222 | 224/228 |
| | | HEX | 223/227 | 227/247 | 235/247 |
| | | NED | 225/233 | 225/227 | 227/227 |
| | | NED | 171/173 | 171/171 | 173/173 |
| | | FAM | 177/186 | 177/184 | 186/188 |
| | | HEX | 208/208 | 208/208 | 206/208 |
| | | FAM | 143/153 | 143/147 | 143/143 |
| | | NED | 203/207 | 195/203 | 203/209 |
| | | FAM | 135/137 | 135/137 | 146/150 |
| | | FAM | 330/330 | 296/310 | 314/316 |
| | | FAM | 173/194 | 182/194 | 215/218 |
| | | HEX | 336/336 | 336/339 | 336/345 |
| | | NED | 258/264 | 240/242 | 254/258 |
| | | FAM | 280/282 | 276/282 | 270/280 |
| | | NED | 291/295 | 291/295 | 291/305 |
| | | HEX | 196/200 | 196/200 | 196/196 |

### REFERENCES:

Bassil N. V., Botta R., Mehlenbacher S. A. 2005a. Microsatellite markers in hazelnut: Isolation, characterization and cross-species amplification. J. Amer. Soc. Hort. Sci. 130:543-549.
Bassil N. V., Botta R., Mehlenbacher S. A. 2005b. Additional microsatellite markers of the European hazelnut. Acta Hort. 686:105-110.
Boccacci P., Akkak A., Bassil N. V., Mehlenbacher S. A., Botta R. 2005. Characterization and evaluation of microsatellite loci in European hazelnut (C. avellana) and their transferability to other Corylus species. Molec. Ecol. Notes 5:934-937.
Boccacci P., Akkak, A. and Botta, R. 2006. DNA typing and genetic relations among European hazelnut (Corylus avellana L.) cultivars using microsatellite markers. Genome 49:598-611.
Gökirmak T., Mehlenbacher S. A., Bassil N. V. 2009. Characterization of European hazelnut (Corylus avellana) cultivars using SSR markers. . Genetic Resources and Crop Evolution 56:147-172.
Gürcan, K., S. A. Mehlenbacher and V. Erdogan. 2010a. Genetic diversity in hazelnut cultivars from Black Sea countries assessed using SSR markers. Plant Breeding (available on-line doi :10. 1111/j. 1439-0523. 2009. 01753. x).
Gürcan, K., S. A. Mehlenbacher, N. V. Bassil, P. Boccacci, A. Akkak and R. Botta. 2010b. New microsatellite markers for Corylus avellana from enriched libraries. Tree Genetics and Genomes (available on-line as DOI 10.1007/s11295-010-0269-y).
Gürcan, K. and S. A. Mehlenbacher. 2010. Development of microsatellite marker loci for European hazelnut (Corylus avellana L.) from ISSR fragments. Molecular Breeding (available on-line).

We claim:

1. A new and distinct cultivar of Corylus plant named ‘Dorris’, as illustrated and described.


SEQUENCE LISTING (48 sequences)

SEQ ID NO: 1 - DNA, 20 bp, Artificial Sequence
  Synthetic polynucleotide
cacacgccttgtcactcttt

SEQ ID NO: 2 - DNA, 20 bp, Artificial Sequence
  Synthetic polynucleotide
cccctttcacatgtttgctt

SEQ ID NO: 3 - DNA, 20 bp, Artificial Sequence
  Synthetic polynucleotide
tggcagagctttgtcagctt

SEQ ID NO: 4 - DNA, 20 bp, Artificial Sequence
  Synthetic polynucleotide
gcagtggaggattgctgact

SEQ ID NO: 5 - DNA, 20 bp, Artificial Sequence
  Synthetic polynucleotide
cactcataccgcaaactcca

SEQ ID NO: 6 - DNA, 20 bp, Artificial Sequence
  Synthetic polynucleotide
atggcttttgcttcgttttg

SEQ ID NO: 7 - DNA, 29 bp, Artificial Sequence
  Synthetic polynucleotide
tgcctctgcagttagtcatcaaatgtagg

SEQ ID NO: 8 - DNA, 25 bp, Artificial Sequence
  Synthetic polynucleotide
cgccatataattgggatgcttgttg

SEQ ID NO: 9 - DNA, 24 bp, Artificial Sequence
  Synthetic polynucleotide
gtaggtgcacttgatgtgctttac

SEQ ID NO: 10 - DNA, 25 bp, Artificial Sequence
  Synthetic polynucleotide
aacaccatattgagtctttcaaagc

SEQ ID NO: 11 - DNA, 20 bp, Artificial Sequence
  Synthetic polynucleotide
tccgtgttgagtatggacga

SEQ ID NO: 12 - DNA, 19 bp, Artificial Sequence
  Synthetic polynucleotide
tgtttttggtggagcgatg

SEQ ID NO: 13 - DNA, 19 bp, Artificial Sequence
  Synthetic polynucleotide
agtcggctccccttttctc

SEQ ID NO: 14 - DNA, 20 bp, Artificial Sequence
  Synthetic polynucleotide
gcgatctgacctcatttttg

SEQ ID NO: 15 - DNA, 20 bp, Artificial Sequence
  Synthetic polynucleotide
cctgcatccaggactcatta

SEQ ID NO: 16 - DNA, 20 bp, Artificial Sequence
  Synthetic polynucleotide
gtgcagaggttgcactcaaa

SEQ ID NO: 17 - DNA, 20 bp, Artificial Sequence
  Synthetic polynucleotide
gagagtgcgtcttcctctgg

SEQ ID NO: 18 - DNA, 19 bp, Artificial Sequence
  Synthetic polynucleotide
agcctcacctccaacgaac

SEQ ID NO: 19 - DNA, 21 bp, Artificial Sequence
  Synthetic polynucleotide
ttgccagtgcatactctgatg

SEQ ID NO: 20 - DNA, 20 bp, Artificial Sequence
  Synthetic polynucleotide
accagctctgggcttaacac

SEQ ID NO: 21 - DNA, 20 bp, Artificial Sequence
  Synthetic polynucleotide
ccaagcacgaatgaactcaa

SEQ ID NO: 22 - DNA, 20 bp, Artificial Sequence
  Synthetic polynucleotide
gcgggttctcgttgtacact

SEQ ID NO: 23 - DNA, 20 bp, Artificial Sequence
  Synthetic polynucleotide
caccctcttcaccacctcat

SEQ ID NO: 24 - DNA, 20 bp, Artificial Sequence
  Synthetic polynucleotide
catcccctgttggagttttc

SEQ ID NO: 25 - DNA, 20 bp, Artificial Sequence
  Synthetic polynucleotide
gttcacaggctgttgggttt

SEQ ID NO: 26 - DNA, 20 bp, Artificial Sequence
  Synthetic polynucleotide
cgtgttgctcatgtgttgtg

SEQ ID NO: 27 - DNA, 20 bp, Artificial Sequence
  Synthetic polynucleotide
ggctgacaacacagcagaaa

SEQ ID NO: 28 - DNA, 20 bp, Artificial Sequence
  Synthetic polynucleotide
tcggctagggttagggtttt

SEQ ID NO: 29 - DNA, 20 bp, Artificial Sequence
  Synthetic polynucleotide
agctggttcttcgacattcc

SEQ ID NO: 30 - DNA, 23 bp, Artificial Sequence
  Synthetic polynucleotide
aaactcaaataaaacccctgctc

SEQ ID NO: 31 - DNA, 20 bp, Artificial Sequence
  Synthetic polynucleotide
gttttgcgagctcattgtca

SEQ ID NO: 32 - DNA, 21 bp, Artificial Sequence
  Synthetic polynucleotide
tgtgtgtggtctgtaggcact

SEQ ID NO: 33 - DNA, 24 bp, Artificial Sequence
  Synthetic polynucleotide
tgtatgtacacacggagagagaga

SEQ ID NO: 34 - DNA, 20 bp, Artificial Sequence
  Synthetic polynucleotide
tgaggggaagaggtttgatg

SEQ ID NO: 35 - DNA, 24 bp, Artificial Sequence
  Synthetic polynucleotide
gacccacaaacaataacctatctc

SEQ ID NO: 36 - DNA, 19 bp, Artificial Sequence
  Synthetic polynucleotide
tgggcatcatccaggtcta

SEQ ID NO: 37 - DNA, 21 bp, Artificial Sequence
  Synthetic polynucleotide
attttccgcagataatacagg

SEQ ID NO: 38 - DNA, 25 bp, Artificial Sequence
  Synthetic polynucleotide
gtttccagatctgcctccatataat

SEQ ID NO: 39 - DNA, 18 bp, Artificial Sequence
  Synthetic polynucleotide
aggcatcagttcatccaa

SEQ ID NO: 40 - DNA, 21 bp, Artificial Sequence
  Synthetic polynucleotide
ggaaggtgagagaaatcaagt

SEQ ID NO: 41 - DNA, 18 bp, Artificial Sequence
  Synthetic polynucleotide
aaggcggcactcgctcac

SEQ ID NO: 42 - DNA, 21 bp, Artificial Sequence
  Synthetic polynucleotide
gaacaactgaagacagcaaag

SEQ ID NO: 43 - DNA, 30 bp, Artificial Sequence
  Synthetic polynucleotide
agaactccgactaataatcctaacccttgc

SEQ ID NO: 44 - DNA, 28 bp, Artificial Sequence
  Synthetic polynucleotide
gagggagcaagtcaaagttgagaagaaa

SEQ ID NO: 45 - DNA, 29 bp, Artificial Sequence
  Synthetic polynucleotide
tggaggaagttttgaatggtagtagagga

SEQ ID NO: 46 - DNA, 28 bp, Artificial Sequence
  Synthetic polynucleotide
aaagcaactcatagctgaagtccaatca

SEQ ID NO: 47 - DNA, 20 bp, Artificial Sequence
  Synthetic polynucleotide
tggcgttgcaacatattctc

SEQ ID NO: 48 - DNA, 25 bp, Artificial Sequence
  Synthetic polynucleotide
gccatctttagaaagttcgatacag